(19) United States
(12) Patent Application Publication
(10) Pub. No.: US 2014/0271911 A1
(43) Pub. Date: Sep. 18, 2014

(54) LITHIUM AND A BETA-SECRETASE INHIBITOR FOR THE TREATMENT OF ALZHEIMER'S DISEASE

(71) Applicant: James Wallace, Staten Island, NY (US)

(72) Inventor: James Wallace, Staten Island, NY (US)

(21) Appl. No.: 14/294,158

(22) Filed: Jun. 3, 2014

Related U.S. Application Data
(60) Provisional application No. 61/970,891, filed on Mar. 26, 2014.

(51) Int. Cl. A61K 33/42; A61K 45/06; A61K 33/00

(52) U.S. Cl. CPC A61K 33/42; A61K 33/00; A61K 45/06
USPC 424/601; 424/715; 424/722

(57) ABSTRACT

This invention is a pharmaceutical composition comprising of lithium as an active ingredient, to decrease intracellular calcium ion concentrations, and a beta-secretase inhibitor as an active ingredient, to reduce beta-secretase activity; for the treatment and prevention of disease, including disease characterized by the abnormal cleavage of amyloid precursor protein.

### CROSS-REFERENCE TO RELATED APPLICATIONS

This present application claims the benefit of U.S. Provisional Application No. 61/970,891 filed on Mar. 26, 2014.

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM, LISTING COMPACT DISC APPENDIX

Not Applicable

### BACKGROUND

Intracellular calcium dysregulation (i.e. chronically elevated concentrations of intracellular cytoplasmic calcium ions) has been characterized as an early, or a proximal occurrence in disease, including disease characterized by the abnormal cleavage of amyloid precursor protein such as Alzheimer's disease; and it is generally accepted that intracellular calcium dysregulation (i.e. chronically elevated concentrations of intracellular cytoplasmic calcium ions) can accelerate the progression of disease (including disease characterized by the abnormal cleavage of amyloid precursor protein) and have an adverse effect on the viability of living tissue (see Querfurth H. W. and Selkoe D. J., Biochemistry, vol. 33, 1994, 4550-4561; Berridge M. J. et al., Nature, vol. 395, 1998, 645-648; Stutzmann G. E., Neuroscientist, vol. 13, 2007, 546-559; Bezprozvanny I. and Mattson M. P., Trends Neurosci., vol. 31, 2008, 454-463; Lopez J. R. et al., J. Neurochem., vol. 105, 2008, 262-271; Demuro A. et al., J. Biol. Chem., vol. 285, 2010, 12463-12468; Berridge M. J., Neurochem. Res., vol. 36, 2011, 1149-1156; Chakroborty S. et al., Sci. China Life Sci., vol. 54, 2011, 752-762; Ferreira I. et al., Cell Calcium, vol. 51, 2012, 95-106; Baker K. D. et al., Neurosci. Biobehay. Rev., vol. 37, 2013, 1211-1239; De Caluwé J., Dupont G., J. Theor. Biol., vol. 331, 2013, 12-18; Bezprozvanny I. and Hiesinger P. R., Mol. Neurodegener., vol. 8, 2013, 23; Wallace J., Cell Calcium, vol. 55, 2014, 175-181).

It is generally accepted that Alzheimer's disease is characterized by the formation and deposition of amyloid-beta (“Aβ” or “Abeta”) and neurofibrillary tangles in brain tissue. It is also generally accepted that amyloid-beta formation precedes neurofibrillary tangle formation, and that amyloid-beta formation results from the abnormal cleavage of amyloid precursor protein, a brain protein, by a two step process that involves cleavage by a beta-secretase and then a gamma-secretase, with beta-secretase cleavage being the proximal event to gamma-secretase cleavage (see Hardy J. and Allsop D., Trends Pharmacol. Sci., vol. 12, 1991, 383-388; Cole S. L. and Vassar R., Mol. Neurodegener., vol. 2, 2007, 22; Ghosh A. K. et al., Neurotherapeutics, vol. 5, 2008, 399-408; Querfurth H. W. and LaFerla F. M., N. Engl. J. Med., vol. 362, 2010, 329-344; Vassar R. et al., J. Neurochem., 2014, Accepted Article, doi: dx.doi.org/10.1111/jnc.12715).

Thus, two processes of significance in disease characterized by the abnormal cleavage of amyloid precursor protein are (1) intracellular calcium dysregulation (i.e. chronically elevated concentrations of intracellular cytoplasmic calcium ions) and (2) the cleavage of amyloid precursor protein by beta-secretase.

A pharmaceutical composition, in a single dosage form, comprising of at least two active ingredients, one that reduces intracellular cytoplasmic calcium ion concentrations, and one that inhibits beta-secretase activity; may be useful for the treatment or prevention of disease, including disease characterized by the abnormal cleavage of amyloid precursor protein, such as Alzheimer's disease.

### SUMMARY OF THE INVENTION

This invention relates to the field of pharmacology. Specifically, a pharmaceutical composition in a single dosage form comprising of at least two active ingredients: (1) lithium, as an active ingredient, to decrease intracellular cytoplasmic calcium ion concentrations, and (2) a beta-secretase inhibitor, as an active ingredient, to reduce beta-secretase cleavage; for the treatment and/or the prevention of conditions or diseases including mild cognitive impairment and Alzheimer's disease, in which a reduction in intracellular cytoplasmic calcium ion concentrations and inhibition of beta-secretase enzyme activity is desired. This invention is also directed to the treatment of non-human mammals in which a reduction in intracellular cytoplasmic calcium ion concentrations and inhibition of beta-secretase enzyme activity is desired. Advantages of this invention over alternatives (e.g. taking the two active ingredients separately in two separate dosage forms) include improved management of medications, a reduced pill burden, convenient dosing, simplified treatment, and improved adherence.

### DETAILED DESCRIPTION OF THE INVENTION

The terms “individual,” “subject,” and “patient,” are used interchangeably herein to refer to a mammal, and can encompass a human or a non-human mammal.

The singular forms “a,” “and,” and “the” as used herein include plural referents unless the context clearly dictates otherwise.

The term “single dosage form” is used herein to refer to a single dose wherein all active and inactive ingredients are combined in a suitable system, such that the patient or person administering the drug to the patient can open a single container or package with the entire dose contained therein. Typical examples of single dosage forms are tablets or capsules for oral administration, single dose vials for injection, or suppositories for rectal administration. This aforementioned list of single dosage forms is not intended to be limiting in any way, but merely to represent typical examples of single dosage forms.

The term “pharmaceutically acceptable carrier” is used herein to refer to a carrier that is useful in preparing a pharmaceutical composition that is generally safe, non-toxic and neither biologically nor otherwise-undesirable, and is acceptable for veterinary use as well as human pharmaceutical use. A “pharmaceutically acceptable carrier” as used in the specification and claims can include both one and more than one such carrier. By “pharmaceutically acceptable” it is meant the carrier must be compatible with the other ingredients of the formulation and not deleterious to the recipient thereof.

The terms “administration of” or “administering a” pharmaceutical composition should be understood to mean providing a pharmaceutical composition to an individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “treatment” or “treating” means any administration of a pharmaceutical composition of this invention to obtain a desired pharmacologic and/or physiologic effect. The effect may be prophylactic in terms of completely or partially preventing a disease or symptom thereof, and/or may be therapeutic in terms of a partial or complete cure for a disease and/or adverse affect attributable to the disease. Treatment includes (a) inhibiting the disease in the subject that is experiencing or displaying the pathology or symptomatology of the diseased (i.e., arresting further development of the pathology and/or symptomatology), or (b) ameliorating the disease in the subject that is experiencing or displaying the pathology or symptomatology of the diseased (i.e., reversing the pathology and/or symptomatology).

Pharmaceutical compositions of this invention may be prepared by any of the methods well known in the art of pharmacy.

Pharmaceutical compositions of this invention encompass any compositions made by admixing the active ingredients and a pharmaceutically acceptable carrier. The carrier may take a wide variety of forms depending on the form of preparation desired for administration, e.g., oral or parenteral (including intravenous). Thus, the pharmaceutical composition of the invention can be presented as discrete units suitable for oral administration such as capsules, cachets or tablets each containing a predetermined amount of the active ingredients. Further, the composition can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the composition may also be administered by controlled release means and/or delivery devices. The foregoing list is illustrative only and is not intended to be limiting in any way.

Pharmaceutical compositions of this invention intended for oral use may contain one or more agents selected from the group consisting of sweetening agents, flavoring agents, coloring agents and preserving agents in order to provide pharmaceutically elegant and palatable preparations. Tablets may contain a composition of the invention in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. A tablet may be prepared by compression or molding, optionally with one or more accessory ingredients or adjuvants. Compressed tablets may be prepared by compressing, in a suitable machine, a compound of the invention in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of this invention for oral use may also be presented as hard gelatin capsules wherein the compound of the invention is mixed with an inert solid diluent, for example, calcium carbonate, calcium phosphate or kaolin, or as soft gelatin capsules wherein the compound of the invention is mixed with water or an oil medium, for example peanut oil, liquid paraffin, or olive oil.

Pharmaceutical compositions of this invention include aqueous suspensions, which contain the active materials in admixture with excipients suitable for the manufacture of aqueous suspensions. In addition, oily suspensions may be formulated by suspending the compound of the invention in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

Pharmaceutical compositions of this invention can be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and should be preserved against the contaminating action of microorganisms such as bacteria and fungi.

Pharmaceutical compositions of this invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound of the invention, to produce a cream or ointment having a desired consistency.

Pharmaceutical compositions of this invention can also be in a form suitable for rectal administration wherein the carrier is a solid. Suitable carriers include cocoa butter and other materials commonly used in the art.

The term “lithium-ions” (known as “Li⁺”) is used herein to refer to the ionic form of elemental lithium that is bioactive, and is known to have a pharmacologic and/or physiologic effects on a subject.

The term “lithium-impurities” is used herein to refer to any form of elemental lithium in a pharmaceutical composition that is not an active ingredient (i.e. any form of elemental lithium in a pharmaceutical composition that was not added for the purpose of exerting pharmacological and/or physiological effects on the subject to whom the pharmaceutical composition is intended).

The term “lithium” as used herein without the descriptors “impurities” or “ions”, refers to any form of elemental lithium in a pharmaceutical composition that is an active ingredient as known in the art of pharmacy; “lithium” can refer to a lithium compound, and/or lithium salts, and/or any type of lithium admixture capable of yielding lithium-ions as an active principle in subjects (e.g. lithium aspartate, lithium carbonate, lithium citrate, lithium gluconate, lithium orotate, NPO3—which is a low-dose lithium microemulsion, or any combinations thereof). The foregoing list is illustrative only and is not intended to be limiting in any way.

Lithium-ions have been shown to decrease intracellular cytoplasmic calcium ion concentrations (see Sourial-Bassillious N. et al., Neuroscience, vol. 161, 2009, 1126-1134; Wallace J., Cell Calcium, vol. 55, 2014, 175-181). Multiple mechanisms may account for this action, including (a) the antagonism of N-methyl-D-aspartate receptors (see Nonaka S. et al., Proc. Natl. Acad. Sci. U.S.A., vol. 95, 1998, 2642-2647; Hashimoto R. et al., J. Neurochem., vol. 80., 2002, 589-597) and (b) the inhibition of inositol monophosphatase (see Hallcher L. M. and Sherman W. R., J. Biol. Chem., vol. 255, 1980, 10896-10901; Berridge M. J. et al., Biochem. J., vol. 206, 1982, 587-595).
(a) N-methyl-D-aspartate receptor channels are integrated within the plasma membrane of cells. The antagonism of N-methyl-D-aspartate receptors by lithium-ions impede the inflow of calcium ions from the extracellular space into the cytoplasm through N-methyl-D-aspartate receptor channels (see Nonaka S. et al., Proc. Natl. Acad. Sci. U.S.A., vol. 95, 1998, 2642-2647; Hashimoto R. et al., J. Neurochem., vol. 80, 2002, 589-597).
(b) Inositol monophosphatase is an intracellular enzyme that removes phosphate from intracellular inositol monophosphate to recycle intracellular inositol. The inhibition of inositol monophosphatase by lithium-ions reduces the quantity of intracellular inositol. A reduction in the quantity of intracellular inositol reduces the availability of intracellular inositol for generating intracellular molecules that contain inositol, including intracellular inositol triphosphate. A reduction in the availability of intracellular inositol for generating intracellular compounds that contain inositol reduces the quantity of intracellular inositol triphosphate. Inositol triphosphate receptor channels are integrated within the surface membrane of the endoplasmic reticulum. A reduction in the quantity of intracellular inositol triphosphate decreases inositol triphosphate mediated calcium ion release of endoplasmic reticulum calcium ion stores into the cytoplasm through inositol triphosphate receptor channels (see Batty I. and Nahorski S. R., J. Neurochem., vol. 45, 1985, 1514-1521; Berridge M. J. et al., Cell, vol. 59, 1989, 411-419; Berridge M. J., Nature, vol. 361, 1993, 315-325; Bezprozvanny I., Cell Calcium, vol. 38, 2005, 261-272; Foskett J. K., Pflugers. Arch., vol. 460, 2010, 481-494; Fedorenko O. A. et al., Eur. J. Pharmacol., 2014, Accepted Article, doi: dx.doi.org/10.1016/j.ejphar.2013.10.074; Shilling D. et al., J. Neurosci. 2014, vol. 34, 6910-6923).

“Beta-secretase” (known as “β-secretase” or “β-site amyloid precursor protein-cleaving enzyme” or “beta-site amyloid precursor protein-cleaving enzyme” or “BACE”) refers to enzymes that are also known as β-site cleaving enzyme types 1 and 2 (“BACE1”, and “BACE2”—a homolog of BACE1) (see Vassar R. et al., Science, vol. 286, 1999, 735-741; Yan R. et al., Nature, vol. 402, 1999, 533-537; Bennett B. D. et al., J. Biol. Chem., 2000, vol. 275, 20647-20651; Farzan M. et al., 2000, Proc. Natl. Acad. Sci. U.S.A., vol. 97, 2000, 9712-9717; Vassar R. et al., J. Neurochem., 2014, Accepted Article, doi: dx.doi.org/10.1111/jnc.12715). It is generally accepted that beta-secretase type 1 (i.e. BACE1) cleaves amyloid precursor protein at an area on amyloid precursor protein characterized as the beta-secretase cleavage site (see Querfurth H. W. and LaFerla F. M., N. Engl. J. Med., vol. 362, 2010, 329-344; Vassar R. et al., J. Neurochem., 2014, Accepted Article, doi: dx.doi.org/10.1111/jnc.12715).

A beta-secretase inhibitor is a compound that impedes the activity of beta-secretase (see Cole S. L. and Vassar R., Mol. Neurodegener., vol. 2, 2007, 22; Ghosh A. K. et al., Neurotherapeutics, vol. 5, 2008, 399-408; Querfurth H. W. and LaFerla F. M., N. Engl. J. Med., vol. 362, 2010, 329-344; Vassar R. et al., J. Neurochem., 2014, Accepted Article, doi: dx.doi.org/10.1111/jnc.12715). This includes beta-secretase inhibitors either in or having been in clinical trials (e.g. AZD3293, AZD3839, CTS-21166, E2609, HPP854, LY2886721, LY2811376, MK-8931, PF-05297909, RG7129, SCH 1359113, TAK-070) and/or, preclinical trials (e.g. GRL-8234, MBI-3); and/or generic equivalents, and/or any other compounds or molecules in which inhibition of beta-secretase activity has been determined and demonstrated by methodology known in the art. A “beta-secretase inhibitor” as used in the specification and claims can include both one beta-secretase inhibitor and more than one beta-secretase inhibitor. The foregoing list of beta-secretase inhibitors is illustrative only and is not intended to be limiting in any way.

Beta-secretase types 1 and 2 (i.e. BACE1, BACE2) may have activity on substrates in addition to or other than amyloid precursor protein, and examples of candidate substrates include neurexin 1 alpha, peptidyl-amidating monooxygenase, type I transmembrane protein, premelanosome protein, seizure 6-like protein, and insulin-like growth factor 2 receptor (for an expanded list of candidate substrates of beta-secretase, see Vassar R. et al., J. Neurochem., 2014, Accepted Article, doi: dx.doi.org/10.1111/jnc.12715). The possible effect of beta-secretase on substrates in addition to or other than amyloid precursor protein does not limit the intended use of this invention, which is for treating disease in which a reduction in intracellular cytoplasmic calcium ion concentrations and inhibition of beta-secretase activity is desired.

The present invention provides a pharmaceutical composition, in a single dosage form, comprising of at least two active ingredients: (a) lithium, to decrease intracellular calcium ion concentrations and (b) a beta-secretase inhibitor, to reduce beta-secretase activity; along with a pharmaceutically acceptable carrier, so that the composition will include at least:
(a) a lithium compound, and/or lithium salts, and/or any type of lithium admixture capable of yielding lithium-ions as an active principle in subjects (e.g. lithium aspartate, lithium carbonate, lithium citrate, lithium gluconate, lithium orotate, NPO3—which is a low-dose lithium microemulsion, or any combination thereof). The foregoing list is illustrative only and is not intended to be limiting in any way.
(b) a beta-secretase inhibitor or beta-secretase inhibitors; which can include a beta-secretase inhibitor either in or having been in clinical trials (e.g. AZD3293, AZD3839, CTS-21166, E2609, HPP854, LY2886721, LY2811376, MK-8931, PF-05297909, RG7129, SCH1359113, TAK-070) and/or, preclinical trials (e.g. GRL-8234, MBI-3); and/or generic equivalents, and/or any other compound or molecule in which inhibition of beta-secretase enzyme activity has been determined and demonstrated by methodology known in the art. The foregoing list of beta-secretase inhibitors is illustrative only and is not intended to be limiting in any way.
(c) a pharmaceutically acceptable carrier.

It is generally accepted that mild cognitive impairment represents an initial or prondromal stage of Alzheimer's disease, and that pharmaceutical agents that may prevent Alzheimer's disease might also prevent mild cognitive impairment (see Gauthier S. et al., Lancet., vol. 367, 2006, 1262-1270; Petersen R. C. et al., Arch Neurol., vol. 66, 2009, 1447-1455).

This invention relates to the manufacture or compounding of a pharmaceutical composition for use in the treatment and prevention of mild cognitive impairment, Alzheimer's disease, and other conditions or diseases that may be treated and/or prevented by a reduction in intracellular cytoplasmic calcium ion concentrations and inhibition of beta-secretase activity; such as Down's syndrome, cerebrovascular dementia, progressive supranuclear palsy (“PSP”), other frontotemporal dementias, Lewy body dementia, Huntington's disease, Parkinson's disease, Parkinson's disease dementia, cerebral amyloid angiopathy (“CAA”), hereditary cerebral hemorrhage with amyloidosis of the Dutch type (“HCHWA-D”), cerebral autosomal dominant arteriopathy with subcortical infarcts and leukoencephalopathy (“CADASIL”), adult metochromatic leukodytrophy, Binzwanger disease, adult-onset adrenoleukodystrophy, other leukodystrophies, Creutzfeld-Jakob disease, prion disorders, HIV associated dementia (“HAD”), amyotrophic lateral sclerosis (“ALS”), spinocerebellar ataxia, head trauma, traumatic brain injury (“TBI”), chronic traumatic encephalopathy (“CTE”), stroke, genetic variants associated with an increased incidence and/or prevalence of dementia such as familial Alzheimer's disease mutations (e.g. mutations in amyloid precursor protein, presenilin 1, presenilin 2) and Apolipoprotein E (“ApoE4”) mutations, pancreatitis, inclusion body myositis, other peripheral amyloidoses, diabetes, and atherosclerosis. The foregoing list is illustrative only and is not intended to be limiting in any way.

The subject or patient to whom the present invention is intended for is a human being, male or female, in whom a reduction in intracellular cytoplasmic calcium ion concentrations and inhibition of beta-secretase enzyme activity is desired; but may also encompass other mammals, such as dogs, cats, mice, rats, cattle, horses, sheep, rabbits, monkeys, chimpanzees or other apes or primates, in which a reduction in intracellular cytoplasmic calcium ion concentrations and inhibition of beta-secretase enzyme activity is desired.

The term “pharmaceutically acceptable lithium-impurities” as used herein means quantities of lithium-impurities in pharmaceutical products that are within the approximate limits defined by The United States Pharmacopeial Convention (“USP”) and The International Conference on Harmonisation of Technical Requirements for Registration of Pharmaceuticals for Human Use (“ICH”). The USP lists the upper limit for daily oral consumption of lithium-impurities at 600 micrograms (“ug”) per day (see http://www.usp.org/sites/default/files/usp—pdf/EN/USPNF/key-issues/2009-04-22MetalImpuritiesCommentDigestpdf) and the ICH lists the Permitted Daily Exposure of lithium-impurities at 780 ug oral, 390 ug parenteral, and 25 ug inhaled (see http://www.fda.gov/downloads/Drugs/GuidanceComplianceRegulatoryInformation/Guidances/UCM371025.pdf). A method for calculating the predicted daily consumption of lithium-impurities from a given treatment is to multiply the amount of lithium-impurities in a single dose by the number of intended doses per day.

This invention does not limit methods well known in the art of pharmacy for synthesizing pharmaceutical compounds, including synthesizing beta-secretase inhibitors, that may result in pharmaceutically acceptable lithium-impurities in a pharmaceutical composition. For example, lithium aluminum hydride is a reagent used for synthesizing organic compounds, and pharmaceutically acceptable lithium-impurities may exist in the final preparation of a pharmaceutical composition if lithium aluminum hydride is utilized in the manufacturing process. In summary, pharmaceutically acceptable lithium-impurities may exist in the final preparation of any type of pharmaceutical composition including one which contains a beta-secretase inhibitor when reagents such as lithium aluminum hydride are utilized during synthesis, and this invention is not intended to be limiting of methods well known in the art of pharmacy that may result in pharmaceutically acceptable lithium-impurities.

Amounts of the component active ingredients in this invention may be varied to (a) provide an optimal therapeutic response and (b) to minimize side effects.

General parameters for the amount of lithium in this invention may vary and may depend on FDA recommendations, the age, weight, and medical condition of the subject to whom this invention is intended, the dosing interval, the number of doses administered per day, and the pharmacodynamics and pharmacokinetics of lithium. The foregoing list of parameters for determining the amount of lithium in this invention is illustrative only and is not intended to be limiting in any way.

General parameters for the amount of beta-secretase inhibitor in this invention may vary and may depend on FDA recommendations, the age, weight, and medical condition of the subject to whom this invention is intended, the dosing interval, and the pharmacodynamics and pharmacokinetics of the beta-secretase inhibitor. The foregoing list of parameters for determining the amount of beta-secretase inhibitor in this invention is illustrative only and is not intended to be limiting in any way.

General parameters for the dosing interval of this invention, and the time of day when this invention is to be administered, may vary and may depend on FDA recommendations, the age, weight, and medical condition of the subject for which this invention is intended, and the pharmacodynamics and pharmacokinetics of the component active ingredients. The foregoing list of parameters for determining the dosing interval and the time of day for administering this invention is illustrative only and is not intended to be limiting in any way.

Those skilled in the art will understand that various modifications may be made to the invention without departing from the spirit or scope thereof. Thus, the present invention is intended to encompass all modifications and variations within the scope of the appended claims and their equivalents.

### REFERENCES CITED—OTHER PUBLICATIONS

1—Baker K. D. et al., “The role of intracellular calcium stores in synaptic plasticity and memory consolidation”, Neurosci. Biobehav. Rev., 2013, vol. 37, 1211-1239.
2—Batty I. and Nahorski S. R., “Differential effects of lithium on muscarinic receptor stimulation of inositol phosphates in rat cerebral cortex slices”, J. Neurochem., vol. 45, 1985, 1514-1521.
3—Bennett B. D. et al., “Expression analysis of BACE2 in brain and peripheral tissues”, J. Biol. Chem., 2000, vol. 275, 20647-20651.
4—Berridge M. J., “Inositol trisphosphate and calcium signalling”, Nature, vol. 361, 1993, 315-325.

5—Berridge M. J., “Calcium signalling and Alzheimer's disease”, Neurochem. Res., vol. 36, 2011, 1149-1156.
6—Berridge M. J. et al., “Lithium amplifies agonist-dependent phosphatidylinositol responses in brain and salivary glands”, Biochem. J., vol. 206, 1982, 587-595.
7—Berridge M. J. et al., “Neural and developmental actions of lithium: a unifying hypothesis”, Cell, vol. 59, 1989, 411-419.
8—Berridge M. J. et al., “Calcium—a life and death signal”, Nature, vol. 395, 1998, 645-648.
9—Bezprozvanny I., “The inositol 1,4,5-trisphosphate receptors”, Cell Calcium, vol. 38, 2005, 261-272.
10—Bezprozvanny I. and Hiesinger P. R., “The synaptic maintenance problem: membrane recycling, Ca2+ homeostasis and late onset degeneration”, Mol. Neurodegener., vol. 8, 2013, 23.
11—Bezprozvanny I. and Mattson M. P., “Neuronal calcium mishandling and the pathogenesis of Alzheimer's disease”, Trends Neurosci., vol. 31, 2008, 454-463.
12—Chakroborty S. et al., “Early calcium dysregulation in Alzheimer's disease: setting the stage for synaptic dysfunction”, Sci. China Life Sci., vol. 54, 2011, 752-762.
13—Cole S. L. and Vassar R., “The Alzheimer's disease beta-secretase enzyme, BACE1”, Mol. Neurodegener., vol. 2, 2007, 22.
14—De Caluwé J., Dupont G., “The progression towards Alzheimer's disease described as a bistable switch arising from the positive loop between amyloids and Ca(2+)”, J. Theor. Biol., vol. 331, 2013, 12-18.
15—Demuro A. et al., “Calcium signaling and amyloid toxicity in Alzheimer disease”, J. Biol. Chem., vol. 285, 2010, 12463-12468.
16—Farzan M. et al., “BACE2, a beta-secretase homolog, cleaves at the beta site and within the amyloid-beta region of the amyloid-beta precursor protein”, Proc. Natl. Acad. Sci. U.S.A., vol. 97, 2000, 9712-9717.
17—Fedorenko O. A. et al., “Intracellular calcium channels: Inositol-1,4,5-trisphosphate receptors”, Eur. J. Pharmacol., 2013, Accepted Article, doi: dx.doi.org/10.1016/j.ejphar.2013.10.074.
18—Ferreira I. et al., “Amyloid beta peptide 1-42 disturbs intracellular calcium homeostasis through activation of GluN2B-containing N-methyl-d-aspartate receptors in cortical cultures”, Cell Calcium, vol. 51, 2012, 95-106.
19—Foskett J. K., “Inositol trisphosphate receptor Ca2+ release channels in neurological diseases”, Pflugers. Arch., vol. 460, 2010, 481-494.
20—Gauthier S. et al., “Mild cognitive impairment”, Lancet., vol. 367, 2006, 1262-1270.
21—Ghosh A. K. et al., “beta-Secretase as a therapeutic target for Alzheimer's disease”, Neurotherapeutics, vol. 5, 2008, 399-408.
22—Hallcher L. M. and Sherman W. R., “The effects of lithium ion and other agents on the activity of myo-inositol-1-phosphatase from bovine brain”, J. Biol. Chem., vol. 255, 1980, 10896-10901.
23—Hardy J. and Allsop D., “Amyloid deposition as the central event in the aetiology of Alzheimer's disease”, Trends Pharmacol. Sci., vol. 12, 1991, 383-388.
24—Hashimoto R. et al., “Lithium protection against glutamate excitotoxicity in rat cerebral cortical neurons: involvement of NMDA receptor inhibition possibly by decreasing NR2B tyrosine phosphorylation”, J. Neurochem., vol. 80., 2002, 589-597.
25—Lopez J. R. et al., “Increased intraneuronal resting [Ca2+] in adult Alzheimer's disease mice”, J. Neurochem., vol. 105, 2008, 262-271.
26—Nonaka S. et al., “Chronic lithium treatment robustly protects neurons in the central nervous system against excitotoxicity by inhibiting N-methyl-D-aspartate receptor-mediated calcium influx”, Proc. Natl. Acad. Sci. U.S.A., vol. 95, 1998, 2642-2647.
27—Petersen R. C. et al., “Mild cognitive impairment: ten years later”, Arch Neurol., vol. 66, 2009, 1447-1455.
28—Querfurth H. W. and LaFerla F. M., “Alzheimer's disease”, N. Engl. J. Med., vol. 362, 2010, 329-344.
29—Querfurth H. W. and Selkoe D. J., “Calcium ionophore increases amyloid beta peptide production by cultured cells”, Biochemistry, vol. 33, 1994, 4550-4561.
30—Shilling D. et al., “Suppression of InsP3 Receptor-Mediated Ca2+ Signaling Alleviates Mutant Presenilin-Linked Familial Alzheimer's Disease Pathogenesis”, J. Neurosci. 2014, vol. 34, 6910-6923.
31—Sourial-Bassillious N. et al., “Glutamate-mediated calcium signaling: a potential target for lithium action”, Neuroscience, vol. 161, 2009, 1126-1134.
32—Stutzmann G. E., “The pathogenesis of Alzheimers disease—is it a lifelong ‘calciumopathy’?”, Neuroscientist, vol. 13, 2007, 546-559.
33—The International Conference on Harmonisation of Technical Requirements for Registration of Pharmaceuticals for Human Use, Guideline For Elemental Impurities, Draft ICH Consensus Guideline—Current Version Step 2b, dated 2013 Jul. 26, [online], [retrieved on 2014 Jun. 02], retrieved from the internet: <URL: http://www.fda.gov/downloads/Drugs/GuidanceComplianceRegulatoryInformation/G uidances/UCM371025.pdf>
34—The U.S. Pharmacopeial Convention, General Chapter on Inorganic Impurities: Heavy Metals Stimuli Article, dated 2009 Apr. 22 [online], [retrieved on 2014 Jun. 2], Retrieved from the internet: <URL: http://www.usp.org/sites/default/files/usp—pdf/EN/USPNF/key-issues/2009-04-22MetalImpuritiesCommentDigestpdf>
35—Vassar R. et al., “Beta-secretase cleavage of Alzheimer's amyloid precursor protein by the transmembrane aspartic protease BACE”, Science, vol. 286, 1999, 735-741.
36—Vassar R. et al., “Function, therapeutic potential and cell biology of BACE proteases: current status and future prospects”, J. Neurochem., 2014, Accepted Article, doi: dx.doi.org/10.1111/jnc.12715
37—Wallace J., “Calcium dysregulation, and lithium treatment to forestall Alzheimer's disease—a merging of hypotheses”, Cell Calcium, vol. 55, 2014, 175-181.
38—Yan R. et al., “Membrane-anchored aspartyl protease with Alzheimer's disease beta-secretase activity”, Nature, vol. 402, 1999, 533-537.

I claim:

1. A pharmaceutical composition, in a single dosage form, comprising of at least two active ingredients: (a) lithium, in a therapeutically effective amount, and (b) a beta secretase inhibitor, in therapeutically effective amount; so that the pharmaceutical composition will include at least:
a) a lithium compound, and/or lithium salts, and/or any type of lithium admixture capable of yielding lithium-ions as an active principle in subjects (e.g. lithium aspartate, lithium carbonate, lithium citrate, lithium gluconate, lithium orotate, NPO3—which is a low-dose lithium microemulsion, or any combination thereof); and
b) a beta-secretase inhibitor or beta-secretase inhibitors; which can include a beta-secretase inhibitor either in or having been in clinical trials (e.g. AZD3293, AZD3839, CTS-21166, E2609, HPP854, LY2886721, LY2811376, MK-8931, PF-05297909, RG7129, SCH 1359113, TAK-070) and/or, preclinical trials (e.g. GRL-8234, MBI-3); and/or generic equivalents, and/or any other compound or molecule in which inhibition of beta-secretase enzyme activity has been determined and demonstrated by methodology known in the art; and
c) a pharmaceutically acceptable carrier.

2. The pharmaceutical composition in claim 1, for use in the manufacture or compounding of a pharmaceutical composition to treat, ameliorate, control, or reduce the risk of Alzheimer's disease.

3. The pharmaceutical composition in claim 1, for use in the manufacture or compounding of a pharmaceutical composition to treat, ameliorate, control, or reduce the risk of mild cognitive impairment.

4. The pharmaceutical composition in claim 1, for use in the manufacture or compounding of a pharmaceutical composition to treat, ameliorate, control, or reduce the risk of Down's syndrome, cerebrovascular dementia, progressive supranuclear palsy (“PSP”), other frontotemporal dementias, Lewy body dementia, Huntington's disease, Parkinson's disease, Parkinson's disease dementia, cerebral amyloid angiopathy (“CAA”), hereditary cerebral hemorrhage with amyloidosis of the Dutch type (“HCHWA-D”), cerebral autosomal dominant arteriopathy with subcortical infarcts and leukoencephalopathy (“CADASIL”), adult metochromatic leukodytrophy, Binzwanger disease, adult-onset adrenoleukodystrophy, other leukodystrophies, Creutzfeld-Jakob disease, prion disorders, HIV associated dementia (“HAD”), amyotrophic lateral sclerosis (“ALS”), spinocerebellar ataxia, head trauma, traumatic brain injury (“TBI”), chronic traumatic encephalopathy (“CTE”), stroke, genetic variants associated with an increased incidence and/or prevalence of dementia such as familial Alzheimer's disease mutations (e.g. mutations in amyloid precursor protein, presenilin 1, presenilin 2) and Apolipoprotein E (“ApoE4”) mutations, pancreatitis, inclusion body myositis, other peripheral amyloidoses, diabetes, and atherosclerosis; and any other condition or disease that may be treated, prevented, ameliorated, or controlled by reducing intracellular cytoplasmic calcium ion concentrations and inhibiting beta-secretase activity.

5. The pharmaceutical composition in claim 1, for use in the manufacture or compounding of a pharmaceutical composition to treat, ameliorate, control, or reduce the risk of disease in non-human mammals.
